(19) United States
(12) Patent Application Publication
(10) Pub. No.: US 2015/0327836 A1
(43) Pub. Date: Nov. 19, 2015

(54) ENDOVASCULAR OCCLUSION DEVICE AND METHOD OF USE

(71) Applicant: UNIVERSITY OF VIRGINIA PATENT FOUNDATION, Charlottesville, VA (US)

(72) Inventors: James R. Stone, Charlottesville, VA (US); John F. Angle, Charlottesville, VA (US); Saher S. Sabri, Charlottesville, VA (US); Ziv J. Haskal, Charlottesville, VA (US)

(21) Appl. No.: 14/714,695

(22) Filed: May 18, 2015

Related U.S. Application Data
(60) Provisional application No. 61/994,559, filed on May 16, 2014.

(51) Int. Cl. A61B 8/08; A61B 8/12; A61B 17/12; A61B 5/06

(52) U.S. Cl. CPC A61B 8/0841; A61B 5/066; A61B 8/12; A61B 17/12109; A61B 17/12136; A61B 2017/1205

(57) ABSTRACT

The disclosed technology relates to endovascular occlusion devices and methods of use. In one embodiment, an endovascular occlusion device includes an occlusion balloon, a catheter for delivering the occlusion balloon to a target area of a subject, one or more pressure sensors coupled to the occlusion balloon and configured to sense a pressure associated with inflation of the occlusion balloon at the target area, and a tracking indicator disposed near the occlusion balloon and configured to indicate, to a non-fluoroscopic detection system, a location of the occlusion balloon relative to the target area.

### CROSS-REFERENCE TO RELATED APPLICATION

This Application claims priority to and benefit under 35 U.S.C §119(e) of U.S. Provisional Patent Application Ser. No. 61/994,559, filed May 16, 2014, which is hereby incorporated by reference herein in its entirety as if fully set forth below.

### BACKGROUND

The disclosed technology generally relates to the treatment of traumatic hemorrhage caused by bodily injury. Traumatic hemorrhage is one of the foremost causes of death in active-duty military service members. Although the widespread use of tourniquets has helped to reduce loss of life from severe lower extremity injury, non-compressible torso hemorrhage continues to carry a high mortality given the relative anatomic inaccessibility of this region. Pelvic bleeding, in particular, can be severe and difficult to control, requiring advanced hospital-based care for definitive treatment. Outside of pelvic binders for pelvic fracture stabilization, which have limited success in hemorrhage control but are of no value in penetrating trauma, there has been little advancement in the control of non-compressible torso bleeding at the lower echelons of care.

Traditionally, temporary control for non-compressible torso hemorrhage has involved thoracotomy with aortic cross-clamping. This technique has been reserved for moribund patients with absent or lost pulses and has an associated high morbidity and mortality. The use of a resuscitative endovascular balloon occlusion of the aorta (REBOA) is an alternative to thoracotomy. For patients with massive pelvic or intra-abdominal hemorrhage who survive transport to an advanced care facility, placement of a temporary occlusion balloon in the infra-renal aorta, proximal to the aortic bifurcation, or the within the descending thoracic aorta may be used to provide time for more definitive treatment through surgical or endovascular methods. The in-hospital procedures stop flow of blood below the level of the balloon until the balloon can be deflated under controlled conditions. Insertion of an occlusive balloon is less invasive than a thoracotomy and can be placed in the unstable patient prior to loss of pulse. Conventionally, fluoroscopic tracking has been used for anatomic localization of the balloon to a target site and to facilitate regulation of the balloon pressure. A medical professional watches expansion of edges of the balloon during inflation and then halts inflation when the edges of the balloon begin to flatten against the wall. Outside of an advanced care setting such as a hospital setting, however, first responders such as military field personnel or EMTs typically will not have fluoroscopic systems readily available.

FIG. 1 shows images of an existing in-hospital aortic occlusion balloon and procedure used to stabilize a patient with massive pelvic hemorrhage. Images A and B are angiographic images demonstrating massive hemorrhage from an iatrogenic laceration of the left common iliac artery following removal of a 26 F sheath that was utilized for placement of an endovascular aortic valve. Image C shows an inflated aortic occlusion balloon that has been introduced from the right common femoral artery, and image D is an angiographic image demonstrating endovascular repair of the traumatic iliac laceration following covered stent placement.

Placement of a temporary occlusion balloon in the aorta is currently performed by highly trained medical professionals under sterile conditions using fluoroscopic guidance. Existing non-fluoroscopic approaches can require the expertise of specialized physicians such as interventional radiologists to interpret subtle tactile cues reflecting appropriate balloon placement and inflation. Proper positioning and confirmation can be time consuming in circumstances where every minute lost dramatically increases a patient's risk of death.

It is with respect to these and other considerations that the various embodiments described below are presented.

### SUMMARY

The disclosed technology generally relates to medical devices and methods of use for occluding blood flow in vital areas of the body after a traumatic injury. Certain aspects of the disclosed technology described herein are directed to endovascular occlusion devices that may be delivered by one or more catheters to a hemorrhage area for the selective placement and deployment of an inflatable balloon. Among other advantages over conventional approaches, embodiments of the disclosed technology provide non-fluoroscopic means that enable non-specialized medical providers to quickly and successfully limit hemorrhage in non-compressible areas until advanced care is available for definitive repair of the underlying injury, thereby decreasing mortality. While certain example embodiments are described herein in the setting of arterial or venous use for the occlusion of hemorrhage in vascular areas, it should be appreciated that the disclosed technology is not limited as such and may alternatively be utilized in other biological systems and to regulate circulation of other bodily fluids.

In one aspect, the disclosed technology relates to an endovascular occlusion device which, in one embodiment, includes an occlusion balloon, a catheter for delivering the occlusion balloon to a target area of a subject, at least one pressure sensor coupled to the occlusion balloon and configured to sense a pressure associated with inflation of the occlusion balloon at the target area, and a tracking indicator disposed near the occlusion balloon and configured to indicate, to a non-fluoroscopic detection system, a location of the occlusion balloon relative to the target area. The target area may be associated with hemorrhage in a vascular area of the subject and may be selected to facilitate control of hemorrhage in the vascular area. The occlusion balloon may be configured to inflate at the target area to occlude blood flow.

The non-fluoroscopic detection system may include an intravascular ultrasound (IVUS) system and the tracking indicator may include an ultrasound probe configured to indicate the location of the occlusion balloon to the IVUS system. The non-fluoroscopic detection system may include an external electromagnetic detection system and the tracking indicator may include a magnetic component configured to indicate the location of the occlusion balloon to the external electromagnetic detection system. The tracking indicator may include a fiber optic component configured to deliver infrared or near-infrared light. The tracking indicator may include at least one component configured for use with a signal processing system to perform at least one of (i) recognizing known waveform signatures corresponding to particular vascular locations and (ii) pressure tracing analysis.

The at least one pressure sensor may be configured to sense a pressure within the occlusion balloon during inflation. The at least one pressure sensor may be configured to sense a pressure exerted on a vascular wall at the target area by inflation of the occlusion balloon. The at least one pressure sensor may include a pressure sensor disposed above the occlusion balloon near the distal end of the catheter that is configured to sense a pressure associated with blood flow above the occlusion balloon in the target area. The at least one pressure sensor may include a pressure sensor disposed below the occlusion balloon that is configured to sense a pressure associated with blood flow below the occlusion balloon in the target area. The at least one pressure sensor may include at least one pressure sensor disposed on an external portion of the occlusion balloon.

In another aspect, the disclosed technology relates to an endovascular occlusion device which, in one embodiment, includes an occlusion balloon and a catheter for delivering the occlusion balloon to a target area associated with hemorrhage in a vascular area of the subject. The target area may be selected to facilitate control of hemorrhage in the vascular area. The occlusion balloon can be configured to inflate at the target area to occlude blood flow. The endovascular occlusion device can further include a plurality of pressure sensors, including at least one pressure sensor coupled to the occlusion balloon and configured to sense a pressure associated with inflation of the occlusion balloon at the target area, and a tracking indicator disposed near the occlusion balloon and configured to indicate, to a non-fluoroscopic detection system, a location of the occlusion balloon relative to the target area.

The non-fluoroscopic detection system may include an intravascular ultrasound (IVUS) system and the tracking indicator may include an ultrasound probe configured to indicate the location of the occlusion balloon to the IVUS system. The non-fluoroscopic detection system may include an external electromagnetic detection system and the tracking indicator may include a magnetic component configured to indicate the location of the occlusion balloon to the external electromagnetic detection system. The tracking indicator may include a fiber optic component configured to deliver infrared or near-infrared light. The tracking indicator may include at least one component configured for use with a signal processing system to perform at least one of (i) recognizing known waveform signatures corresponding to particular vascular locations and (ii) pressure tracing analysis.

The plurality of pressure sensors may include a pressure sensor configured to sense a pressure within the occlusion balloon during inflation. The plurality of pressure sensors may include a pressure sensor configured to sense a pressure exerted on a vascular wall at the target area by inflation of the occlusion balloon. The plurality of pressure sensors may include a pressure sensor disposed above the occlusion balloon near the distal end of the catheter that is configured to sense a pressure associated with blood flow above the occlusion balloon in the target area and/or a pressure sensor disposed below the occlusion balloon that is configured to sense a pressure associated with blood flow below the occlusion balloon in the target area. The plurality of pressure sensors may include at least one pressure sensor disposed on an external portion of the occlusion balloon.

In another aspect, the disclosed technology relates to a method for endovascular occlusion which, in one embodiment, includes delivering an occlusion balloon to a target area of a subject, which includes guiding the occlusion balloon to the target area using non-fluoroscopic detection of the location of a tracking indicator disposed near the occlusion balloon. The method can also include inflating the occlusion balloon at the target area based on at least one pressure associated with the inflation. The at least one pressure can be sensed using at least one pressure sensor coupled to the occlusion balloon.

The target area may be associated with hemorrhage in a vascular area of the subject and may be selected to control hemorrhage in the vascular area. Inflating the occlusion balloon may include inflating the occlusion balloon to a particular inflation pressure to occlude blood flow in the target area. The particular inflation pressure may correspond to the systolic blood pressure.

The tracking indicator may include an ultrasound probe, and detecting the location of the tracking indicator may be performed using an intravascular (IVUS) system. The tracking indicator may include a magnetic component, and detecting the location of the tracking indicator may be performed using an external electromagnetic detection system. The tracking indicator may include a fiber optic component configured to deliver infrared or near-infrared light. The tracking indicator may include at least one component configured for use with a signal processing system, and detecting the location of the tracking indicator may include performing at least one of (i) recognizing known waveform signatures corresponding to particular vascular locations and (ii) pressure tracing analysis.

The at least one pressure sensor may include a pressure sensor configured to sense the pressure within the occlusion balloon during the inflation. The at least one pressure sensor may include a pressure sensor configured to sense the pressure exerted on the vascular wall by the inflation of the occlusion balloon. The at least one pressure sensor may include a pressure sensor disposed above the occlusion balloon near the distal end of the catheter that is configured to sense a pressure associated with blood flow above the occlusion balloon in the target area. The at least one pressure sensor may include a pressure sensor disposed below the occlusion balloon that is configured to sense a pressure associated with blood flow below the occlusion balloon in the target area. The at least one pressure sensor may include at least one pressure sensor disposed on an external portion of the occlusion balloon.

Other aspects and features according to the disclosed technology will become apparent to those of ordinary skill in the art, upon reviewing the following detailed description in conjunction with the accompanying figures.

### BRIEF DESCRIPTION OF THE DRAWINGS

Reference will now be made to the accompanying drawings, which are not necessarily drawn to scale.

FIG. 1 shows images of an existing in-hospital procedure using an aortic occlusion balloon to stabilize a patient with pelvic hemorrhage.

FIG. 2 illustrates an endovascular occlusion device according to one embodiment of the disclosed technology.

FIG. 3 illustrates an endovascular occlusion device according to another embodiment of the disclosed technology.

FIGS. 4A, 4B, and 4C illustrate endovascular stabilization of a lower extremity hemorrhage using a device and method according to embodiments of the disclosed technology.

FIG. 5 illustrates a device and method for endovascular occlusion using intravascular ultrasound (IVUS) guidance in accordance with some embodiments of the disclosed technology.

FIG. 6 shows images of electromagnetic (EM) tracking used for guidance of an occlusion balloon into the distal abdominal aorta, in accordance with an example implementation of some embodiments of the disclosed technology.

FIG. 7 shows images of IVUS used for guidance of an occlusion balloon into the distal abdominal aorta, in accordance with an example implementation of some embodiments of the disclosed technology.

FIG. 8 shows images of the balloon inflation and corresponding pressure measurements in accordance with an example implementation of some embodiments of the disclosed technology.

FIG. 9 shows flush aortogram and pressure transduction measurements above and below an aortic occlusion balloon in a swine model, in accordance with an example implementation of some embodiments of the disclosed technology.

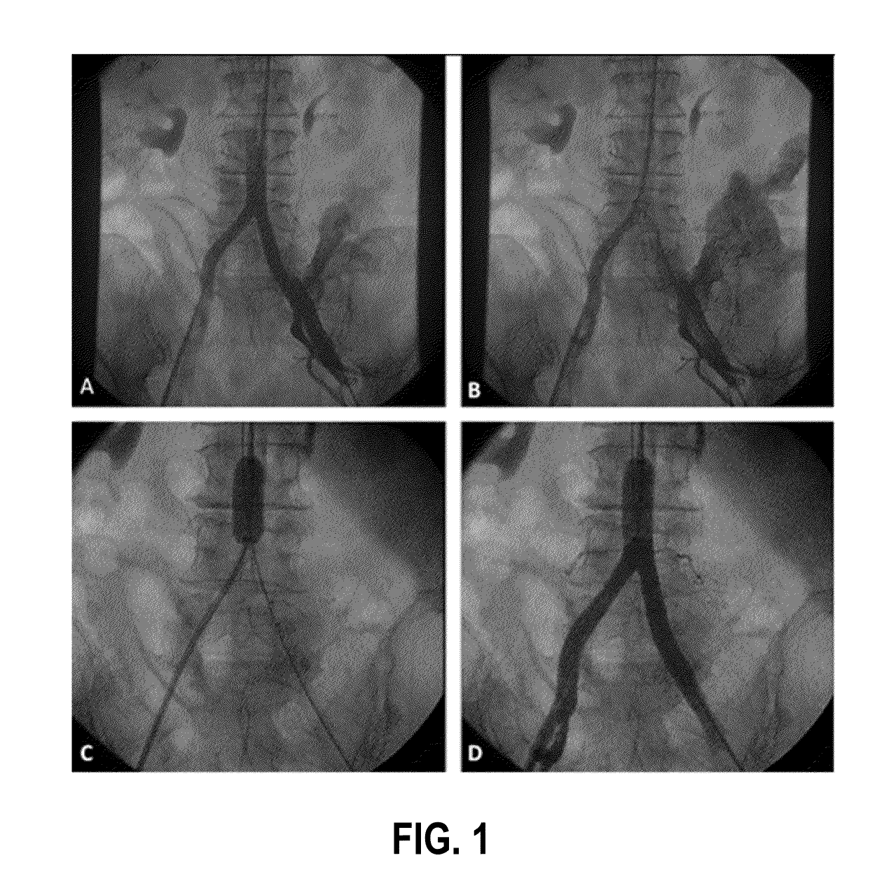

### DETAILED DESCRIPTION

In some example embodiments, the disclosed technology relates to endovascular occlusion devices and methods of use. Although example embodiments of the disclosed technology are explained in detail herein, it is to be understood that other embodiments are contemplated. Accordingly, it is not intended that the disclosed technology be limited in its scope to the details of construction and arrangement of components set forth in the following description or illustrated in the drawings. The disclosed technology is capable of other embodiments and of being practiced or carried out in various ways.

It must also be noted that, as used in the specification and the appended claims, the singular forms “a,” “an” and “the” include plural referents unless the context clearly dictates otherwise. By “comprising” or “containing” or “including” it is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

In the following description, references are made to the accompanying drawings that form a part hereof and that show, by way of illustration, specific embodiments or examples. In referring to the drawings, like numerals represent like elements throughout the several figures.

As discussed herein, a “subject” or “patient” may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to a human (e.g., rat, dog, pig, monkey), etc. It should be appreciated that the subject may be any applicable human patient, for example.

In certain embodiments of the disclosed technology, an aortic occlusion balloon catheter is configured to minimize the size of the arteriotomy required for insertion and uses non-fluoroscopic balloon tracking methods that provide reliable and readily interpretable data concerning location of the balloon relative to vascular anatomic landmarks. Further embodiments provide for the verification of optimal balloon inflation pressure to maximize seal while minimizing aortic wall injury and the integration of pressure and guidance sensors.

Certain compliant balloons capable of occluding the aorta are designed exclusively for the in-hospital setting, which may have been produced for use as an adjunct for the placement of abdominal or thoracic aortic stent grafts for the treatment of aortic aneurysm, dissection, or disruption. The introduction of stent grafts involves the creation of relatively large femoral arteriotomies, and aortic balloons are typically used after stent grafts are deployed to aid with apposition of the proximal, distal, and junctional aspects of these devices. When used in an aortic occlusion procedure, as the balloon expands from the inflation, it experiences reciprocal pressure from a combination of fluid pulses transmitted through the circulatory system by action of the heart (systolic blood pressure) and by contact with the wall. After diligent study, the present inventors have recognized that the balloon should ideally be inflated to a pressure that matches the systolic blood pressure (SBP). If the balloon is inflated past SBP, it may exert too much pressure on the wall, which may result in an aortic wall injury.

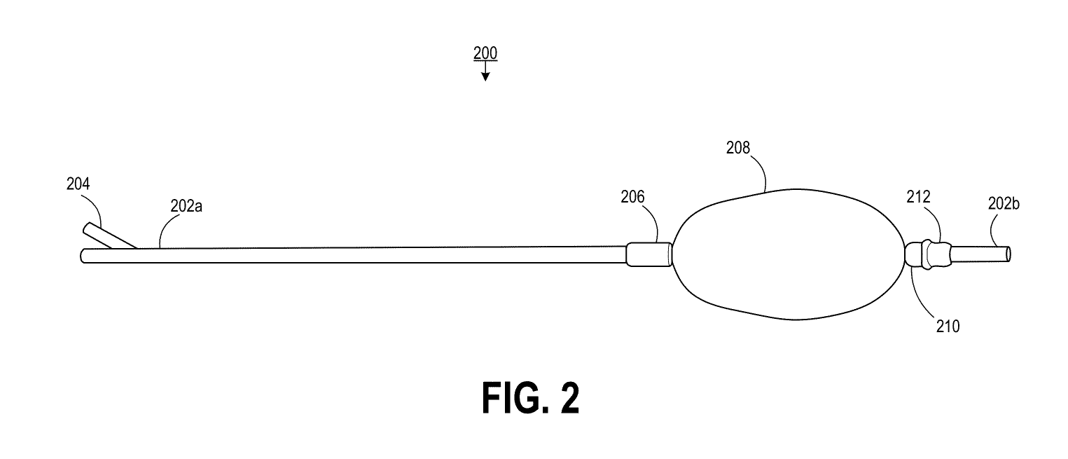

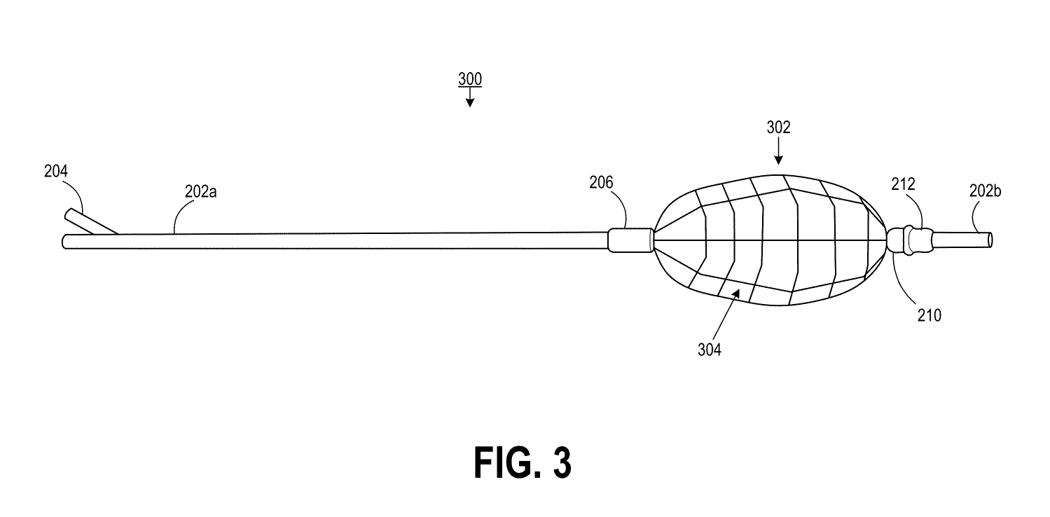

FIGS. 2 and 3 illustrate devices for endovascular occlusion in accordance with embodiments of the disclosed technology. FIG. 2 is a diagram illustrating an endovascular occlusion device 200 with a catheter 202 and inflatable occlusion balloon 208 disposed near the distal end 202b of the catheter 202. The device 200 also includes a plurality of pressure sensors 206 and 210 disposed on either side of the balloon 208, which can be configured to sense pressures associated with inflation of the occlusion balloon 208. The pressure sensors 206 and 210 can sense fluid pressure below and above the balloon 208, respectively. The device 200 can also include a pressure sensor configured to transduce pressure from the inflation lumen (see 204 at proximal end 202a of catheter) for measuring the pressure within the occlusion balloon 208 during inflation.

A tracking indicator 212 can be configured to indicate, to a non-fluoroscopic detection system, a location of the balloon 208 as the balloon 208 is guided to a target area for placement and inflation. The tracking indicator 212 can be configured to indicate the location of the balloon 208 to an external, non-fluoroscopic system. The tracking indicator 212 may include, for example, one or more of an ultrasound probe for communication with an external IVUS system, one or more magnets for communication with an external electromagnetic detection system, or a fiber optic component for delivering infrared or near-infrared light to a detection system. The tracking indicator 212 may additionally or alternatively include one or more components configured for use with a signal processing system to perform recognition of known waveform signatures corresponding to particular vascular locations and/or to perform pressure tracing analysis. When taken together, the pressure sensors 206 and 210 can be used for determining the pressure differential between the respective sides of the balloon. The device 200 may also include a pressure sensor that is configured to sense an inflation pressure of the balloon 208 during inflation and also when the inflated balloon exerts pressure on a vascular wall.

The endovascular device 300 illustrated in FIG. 3 includes similar catheter and pressure sensor components to the device 200 shown in FIG. 2, which are numbered to correspond with the like components in FIG. 2. The device 300 includes a plurality of external pressure sensors 304 disposed in an array arrangement on the external surface of the balloon 302 and configured to measure interactions between the balloon 308 and balloon wall, for example to provide fine feedback concerning the precise interface between the balloon 302 and aortic wall. Each of the embodiments in FIGS. 2 and 3 can feature a multi-lumen design that allows for the integration of the multiple sensors to guide tracking and inflation of the occlusion balloon. In this design, one lumen allows the balloon to travel over a guidewire, and a second lumen is in continuity with the balloon space. The second lumen can be hooked to a syringe or inflation device where a mixture inert gases, saline, or dilute contrast may be used to expand the balloon to a desired diameter. A three-way adapter, such as a 3 way stop cock from Sterimed Medical Devices Pvt. Ltd., allows for a pressure sensor to transduce pressure from the inflation lumen (see 204 at proximal end 202a of catheter 200). Two ports of the adapter can be used for inline flow into the balloon from the inflation device and the third port can be used to trace an intraballoon pressure to compare with a pressure sensor mounted at the distal end of the catheter above the balloon (see 212). The pressure sensors can provide differential and/or absolute pressure measurements. Among other types of pressure sensors, piezoresistive transducers may be used. Pressure sensing wires such as a Radi wire or Aeris wire from St. Jude Medical may be used.

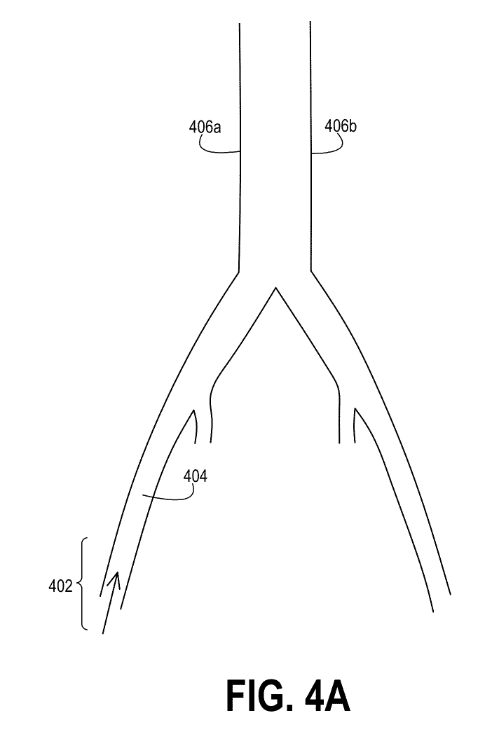

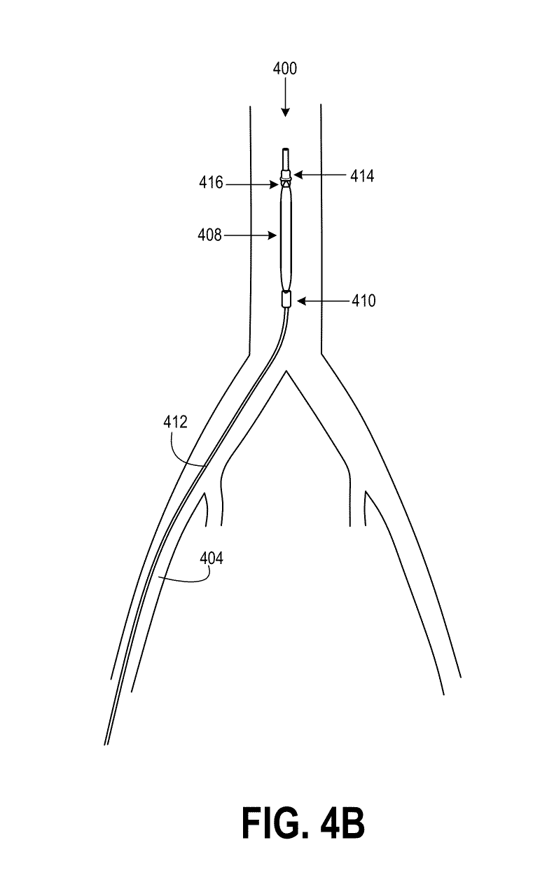

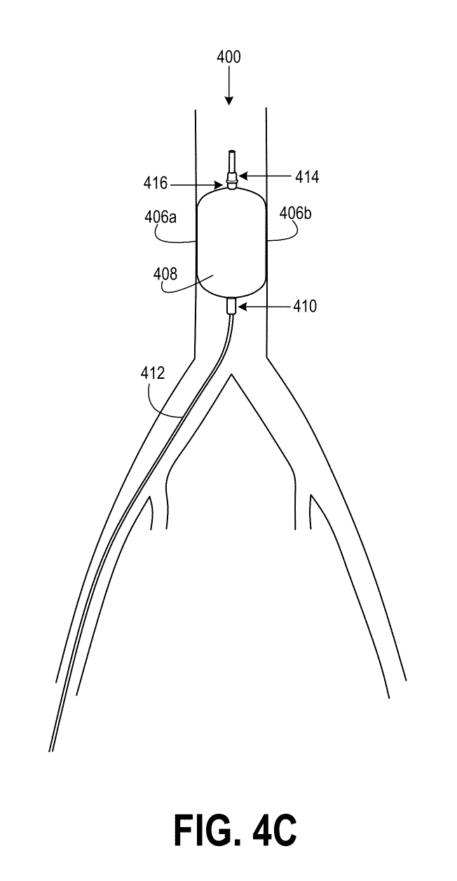

FIGS. 4A-4C illustrate the implementation of devices and methods according to some embodiments of the disclosed technology for endovascular stabilization of massive pelvic or lower extremity hemorrhage. In FIG. 4A, 402 illustrates accessing the common femoral artery 404. FIG. 4B illustrates the placement of an endovascular occlusion device 400 according to an embodiment of the disclosed technology, prior to inflation of an occlusion balloon 408. As shown, the device 400 is configured as a low profile occlusion balloon catheter device with pressure sensors 410, 416 mounted on either side of the occlusion balloon 408 near the distal end of a catheter 412. A balloon tracking sensor 414 (also referred to herein as a “tracking indicator”) is configured such that its location and position can be tracked using one or more of intravascular ultrasound (IVUS), electromagnetic (EM) tip tracking, infrared (IR) or near-infrared tracking, or signal processing localization (SPL).

FIG. 4C shows inflation of the occlusion balloon 408, which may be performed by transducing pressures through the balloon catheter 412 to match systolic blood pressure (SBP) and/or using a balloon-mounted external sensor array (see FIG. 3). Using devices and methods according to some embodiments of the disclosed technology identified herein, optimal balloon inflation protocols may be established and used to identify parameters that allow for maximal balloon wall apposition while avoiding disruption the aortic wall 406 (see left and right portions 406a, 406b). Again with reference to FIG. 4C, once the balloon 408 is advanced to an optimal location, insufflation can be performed until the edges of the balloon 408 begin to flatten, which is indicative of broad contact with the aortic wall 406. Particular care is taken at this point to minimize further inflation to avoid injury to the aortic wall 406.

As briefly described above, balloon location and position can be tracked using one or more of intravascular ultrasound (IVUS), electromagnetic (EM) tracking, infrared (IR) or near-infrared tracking, or signal processing localization (SPL). IVUS allows for the anatomic visualization of vessel wall and limited surrounding tissues from the inside of the vessel lumen and is currently used for a variety of applications, for example in both the arterial and venous systems. A spectrum of catheters is available, which allow for detailed visualization of vessel sizes across a broad range, from coronary artery to aorta or IVC.

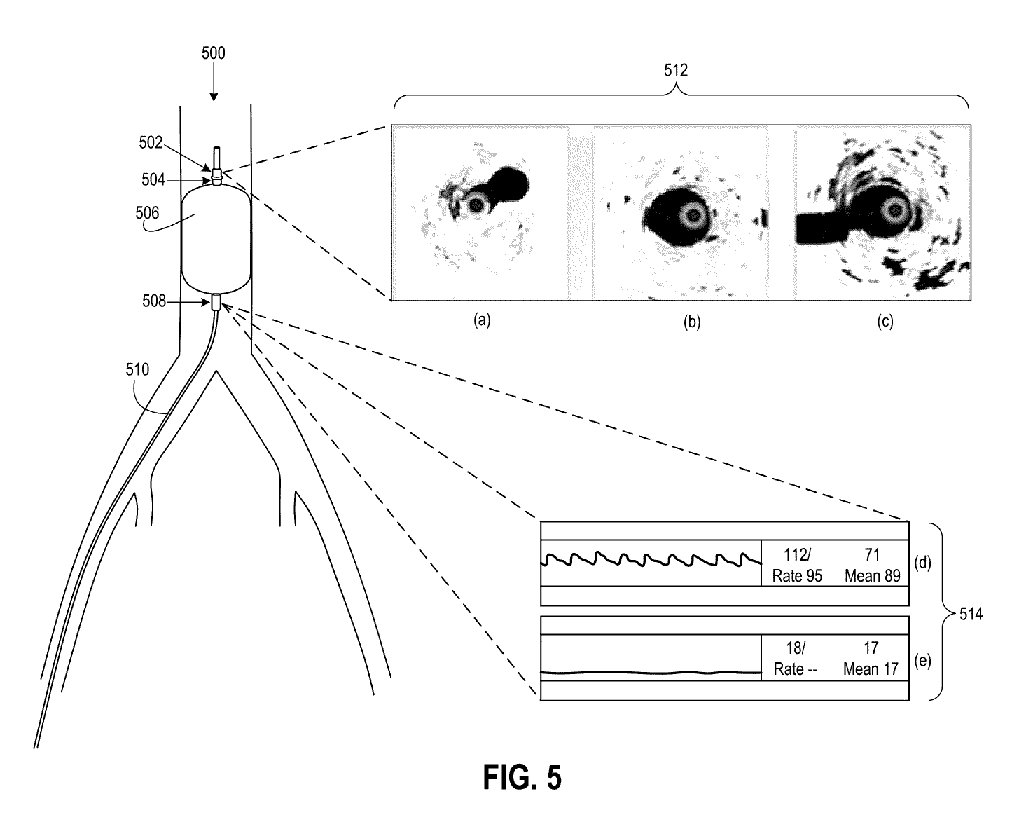

In some embodiments of the disclosed technology described in further detail below with reference to FIG. 5, an endovascular occlusion device can include an ultrasound probe at a distal tip beyond the balloon. The proximal end of the catheter may be attached to computerized ultrasound equipment for medical imaging of a site of interest, for example commercially available Volcano PC IVUS or BSC Polaris systems. In order to visualize an artery or vein, a user can steer a guidewire from outside the body of a subject, through the catheters and into a blood vessel branch to be imaged. Sound waves are emitted from the ultrasound probe, and the catheter may receive and conduct the return echo information out to external computerized ultrasound equipment, which may construct and display real time ultrasound images of a section of the blood vessel surrounding the catheter tip.

EM tracking can utilize components currently used for the placement of peripherally inserted central catheter (PICC) lines at the beside, such as one or more components of the Sherlock system from Bard, Inc. Infrared (IR) or near-infrared light tracking can use fiber optic sensors to deliver near-infrared light using, for example, one or more components of a SonoSite LumenVu catheter guidance system, in which a fiber optic stylet can be used in place of a traditional guide wire to provide visualization and real-time tracking of a catheter tip as it advances through a vessel.

In various embodiments, the disclosed technology may also utilize techniques based on sound wave signal processing and pressure tracing analysis. Devices in accordance with some embodiments may include pressure sensors and/or various ultrasound equipment which may be used to chart out fluid pulses as they move throughout the vascular systems, such as fluid pulses associated with different levels of the aorta. In certain implementations, as a balloon catheter moves further away from the heart and into areas where branch vessels are present, certain waveforms may be recognizable in comparison to known signatures, such that by the identification of these recognizable signatures, the location of a balloon relative to branch vessels can be determined. Various aspects of technology produced by Elcam Medical, for example, can be adapted for localization of a catheter within the venous vasculature based upon pressure tracing characteristics distinct to segments of the upper extremity and superior vena cava. The various tracking means described herein in embodiments of the disclosed technology may be utilized in the abdominal aorta to determine the location of a catheter with respect to the aortic bifurcation and branch vessels.

Aspects of the disclosed technology that implement IVUS components and methods to guide an endovascular occlusion device 500 will now be described with reference to the embodiment shown in FIG. 5. Images (a), (b), and (c) show IVUS-guided balloon positioning used with computer-assisted image interpretation. The IVUS images are acquired using an ultrasound probe 502 mounted proximate the tip of the catheter 510. Real-time images 512 can provide feedback to a care provider to locate an optimal inflation zone. For example, image (a) represents a location that is too low (aortic bifurcation), image (b) represents the optimal inflation zone, and image (c) represents a location that is too high (right renal artery). A pressure transducer 504 can be mounted just inferior to the occlusion balloon 506 to guide inflation and provide direct information 514 on degree of flow reduction below the balloon 506 (see blood pressure readings), where image (d) corresponds to the balloon 506 being deflated and image (e) corresponds to the balloon 506 being inflated.

Some embodiments of the disclosed technology provide for automated image interpretation functions to give indications of when it is acceptable to deploy the occlusion balloon, using a device a having spatial awareness and understanding of expected anatomy. In some embodiments, multiple IVUS sensors can be used to acquire frames of reference to be compared with each other. An image interpretation system according to embodiments of the disclosed technology can be configured to recognize when the catheter has passed major branch vessels (e.g., internal iliac artery, contralateral common iliac artery, inferior mesenteric artery, renal arteries) and provide feedback on catheter advancement or retraction to a desired zone (between the renal arteries and aortic bifurcation, for example). Obtained images may be color coded to further visually represent the various locations, for example by displaying a red background in locations that are too low or too high and a green background in the optimal inflation zone. Further, a device in some embodiments of the disclosed technology may include an accelerometer to determine the motion of the catheter or an interaction between the catheter and the sheath to sense linear and rotational movements.

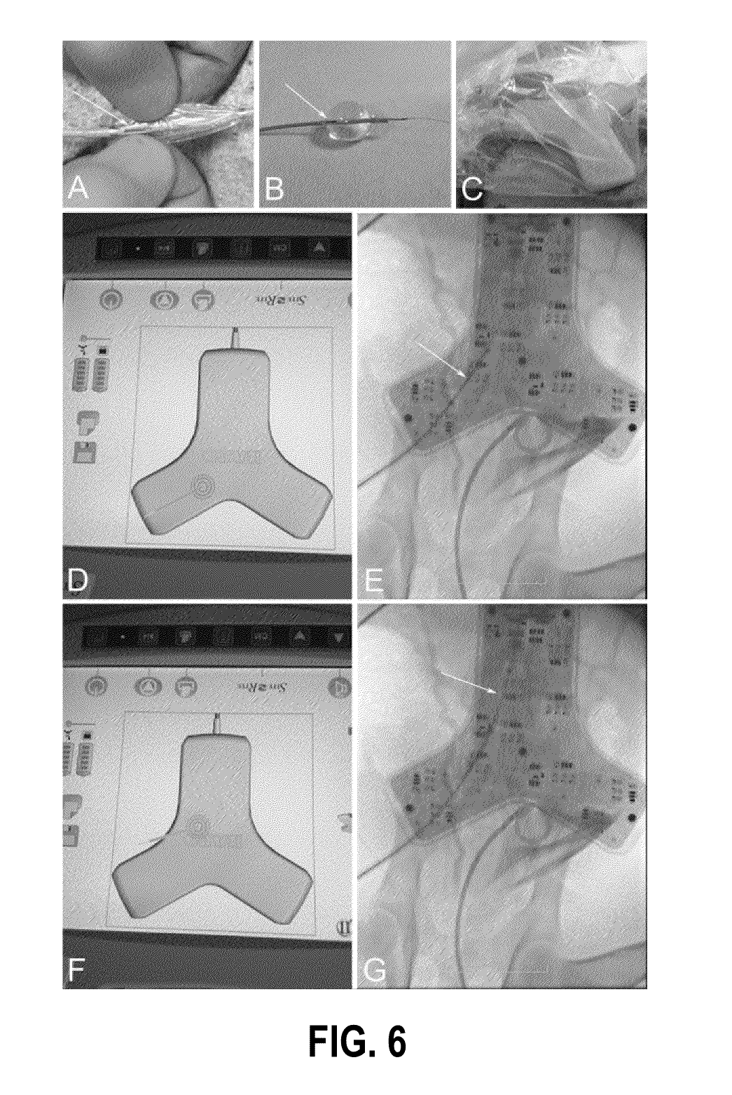

Various aspects of the disclosed technology may be still more fully understood from the following description of some example implementations and corresponding results and the images of FIGS. 6-9. Some experimental data are presented herein for purposes of illustration and should not be construed as limiting the scope of the disclosed technology in any way or excluding any alternative or additional embodiments.

A first example of certain implementations of the disclosed technology and corresponding results will now be described, in which two separate non-fluoroscopic approaches for guidance of an occlusion balloon into the distal abdominal aorta were assessed in swine weighing between 70-100 kg. These methods included: (i) Electromagnetic (EM) tip tracking, and (ii) intravascular ultrasound (IVUS). Balloon locations estimated by EM tip tracking or IVUS were compared with intermittent fluoroscopy to determine the accuracy of each approach.

As shown in further detail in FIG. 6, EM tip tracking was performed by modifying a Bard Sherlock II tip location system (C.R. Bard, Inc., NJ, USA) for use with an aortic occlusion balloon. The magnetic stylette used with this system was inserted into the balloon lumen of a Q50 aortic occlusion balloon (W.L. Gore, DE, USA). The tip tracking element was positioned approximately 1 cm superior to the lower radio-opaque marker of the balloon, as shown in images A and B. This balloon system was inserted through a 10 F sheath located in the common femoral artery (CFA). The detector element from the Bard Sherlock II system was placed over the lower abdomen of swine, as shown in image C. Catheter location indicated by EM tracking (images D and F) was compared with fluoroscopic images (images E and F).

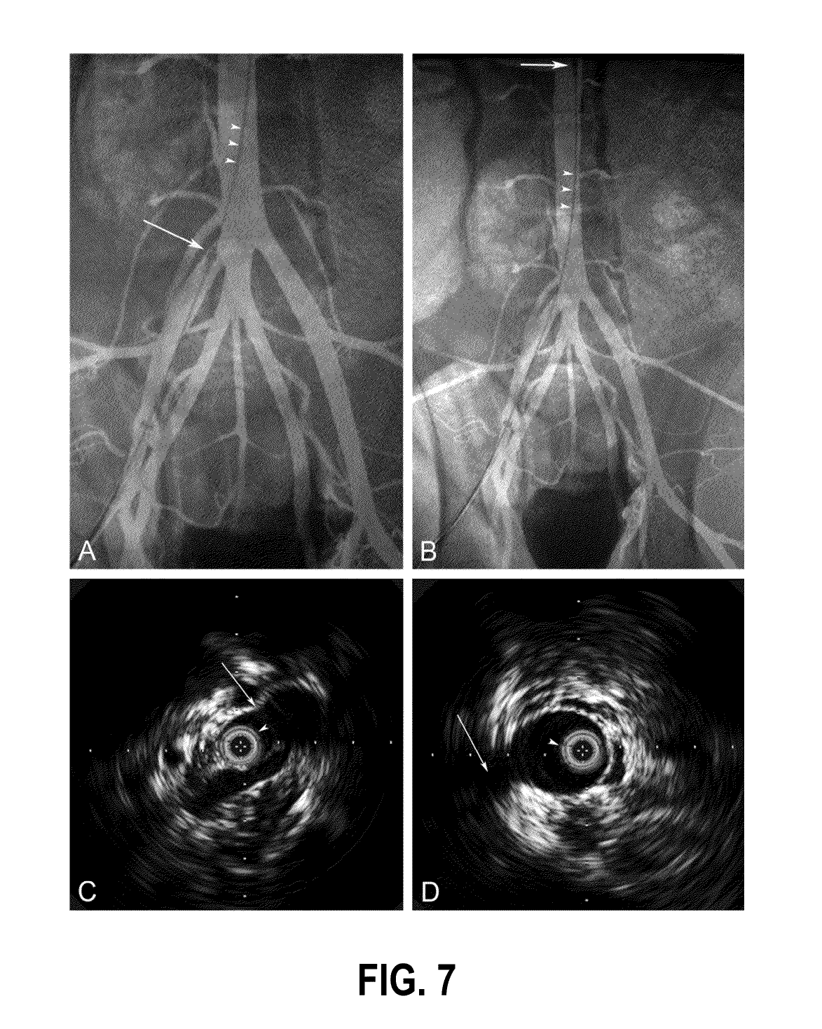

For IVUS, a Visions PV catheter (Volcano Corp, CA, USA) was inserted through a 10 F vascular sheath into the CFA. Location of the probe was estimated in relation to anatomic landmarks and compared with fluoroscopy superimposed on a digital subtraction angiogram through fluoro-fade of the inferior abdominal aorta and pelvis. FIG. 7 shows representative images from the aortic bifurcation (images A and C) and the abdominal aorta at the level of the right renal artery (images B and D).

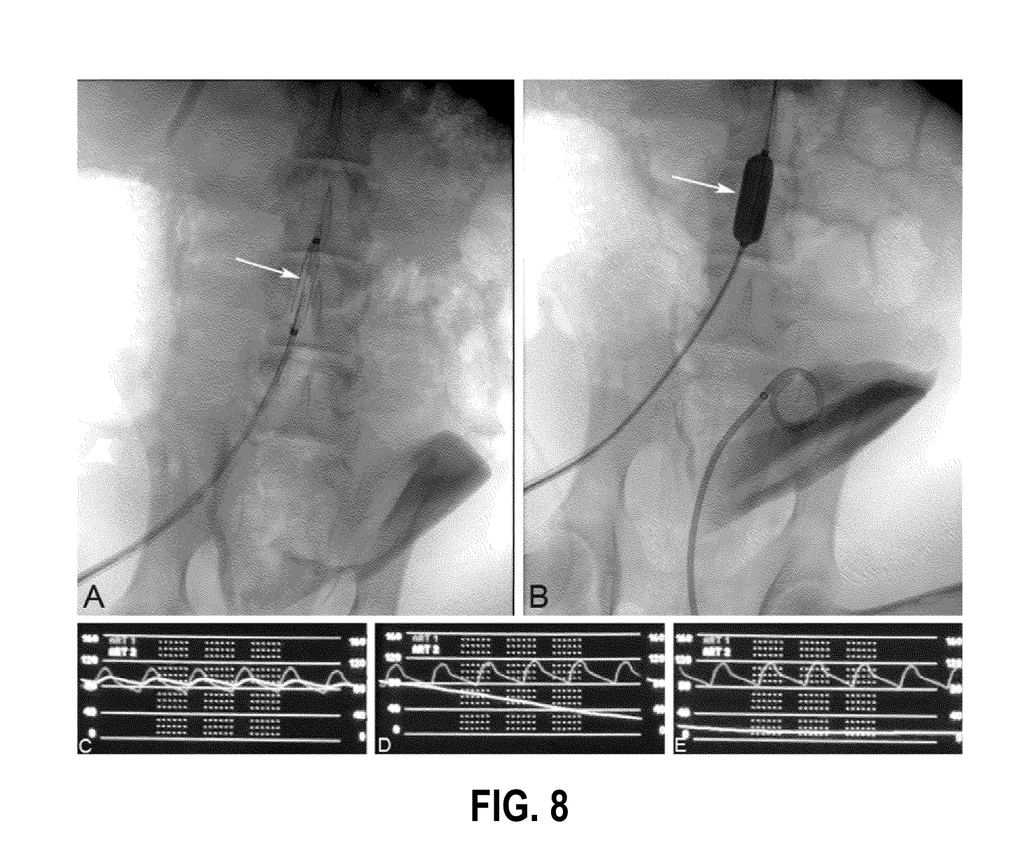

To assess balloon inflation without fluoroscopic guidance, pressure measurements were simultaneously obtained through wire lumen of the Q50 balloon and the CFA vascular sheath following creation of pelvic hemorrhage in a swine model as previously described. Images A and C of FIG. 8 demonstrate appearance of the balloon and pressure measurements prior to inflation. Images B and E of FIG. 8 demonstrate optimal balloon inflation, while image D demonstrates active inflation.

In summary of some results, IVUS proved to be an accurate method for non-fluoroscopic visualization of landmarks required for balloon catheter placement. Trials showed agreement between expected location by IVUS and actual location by fluoroscopy. EM tip tracking was also shown to be a workable option for determining location of the occlusion balloon. Pressure transduction was shown to be effective for determining optimal balloon inflation. Reduction of intra-arterial pressure by 80% in the vascular sheath tracing correlated with optimal balloon inflation by fluoroscopy.

A second example of certain implementations and corresponding results will now be described. A Q50 aortic occlusion balloon (W.L. Gore and Assoc., Inc.) was introduced through a 12 F sheath inserted in the right common femoral artery of swine. A pressure wire (Volcano, Corp., San Diego, Calif.) was introduced through the right common femoral artery sheath adjacent to the balloon and positioned above the balloon. Through the contralateral femoral artery, a 5 F pigtail catheter was inserted and positioned above the balloon. Through this same sheath, a pressure transducer was attached to measure intravascular pressure below the balloon. A three-way adapter was placed between the balloon catheter lumen leur-loc connector and the insufflation device. The side arm of this three-way adapter was connected to a pressure transducer to determine pressures within the balloon during inflation. Under direct fluoroscopic guidance, the balloon was inflated to its optimal diameter based upon both fluoroscopic visualization of the balloon contour and also confirmation of vascular occlusion as determined by a flush aortogram through the pigtail catheter located above the balloon.

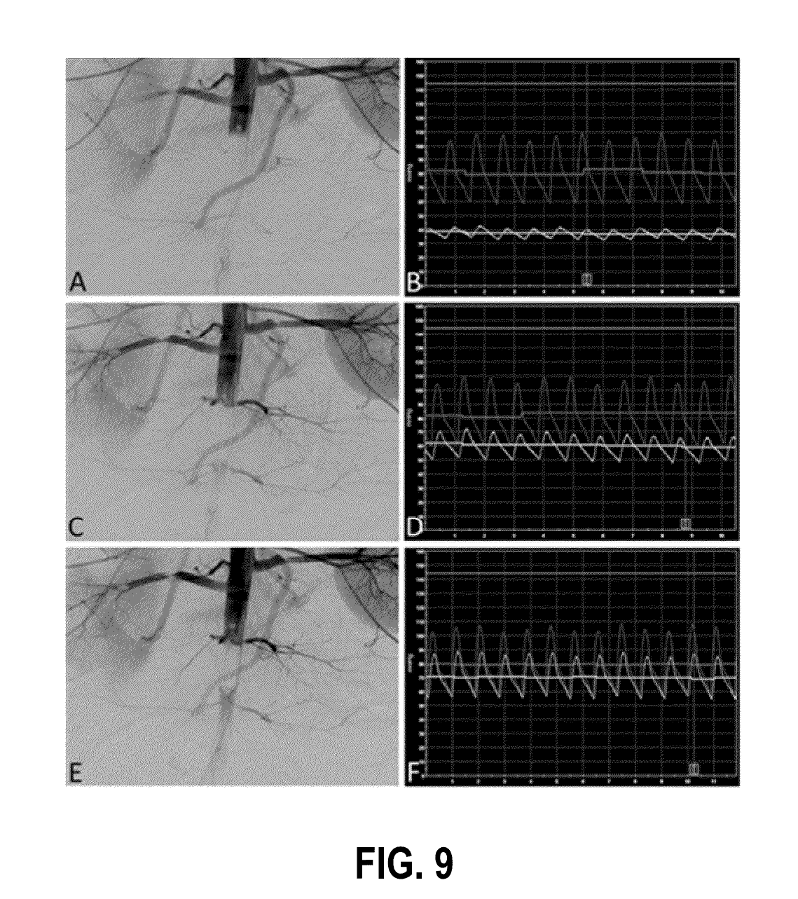

FIG. 9 shows a flush aortogram and pressure transduction measurements above and below an aortic occlusion balloon in the swine model. First the balloon was inflated to match SBP (images A and B). Next, the balloon was deflated to 75% of this maximal value and an aortogram was obtained (images C and D). Finally, the balloon was deflated to 50% of this maximal value (images E and F). Optimal balloon inflation was found to correspond with intraballoon pressures which matched the systolic blood pressure (SBP) measured through the pressure wire positioned above the balloon, that is, the intra-balloon pressures matched with SBP yielded optimal fluoroscopic appearance of balloon and minimized pressures caudal to the balloon. Of note, pressures were not completely abolished, likely to collateral flow through mesenteric and lumbar collaterals. Air was used as the balloon inflation medium.

The specific configurations, choice of materials and the size and shape of various elements can be varied according to particular design specifications or constraints requiring a system or method constructed according to the principles of the disclosed technology. Such changes are intended to be embraced within the scope of the disclosed technology. The presently disclosed embodiments, therefore, are considered in all respects to be illustrative and not restrictive. The scope of the present invention is indicated by the appended claims, rather than the foregoing description, and all changes that come within the meaning and range of equivalents thereof are intended to be embraced therein.

What is claimed is:

1. An endovascular occlusion device, comprising:
an occlusion balloon;
a catheter for delivering the occlusion balloon to a target area of a subject;
at least one pressure sensor coupled to the occlusion balloon and configured to sense a pressure associated with inflation of the occlusion balloon at the target area; and
a tracking indicator disposed near the occlusion balloon and configured to indicate, to a non-fluoroscopic detection system, a location of the occlusion balloon relative to the target area.

2. The endovascular occlusion device of claim 1, wherein the target area is selected to facilitate control of hemorrhage in a vascular area of the subject.

3. The endovascular occlusion device of claim 2, wherein the occlusion balloon is configured to inflate at the target area to occlude blood flow.

4. The endovascular occlusion device of claim 1, wherein the non-fluoroscopic detection system comprises an intravascular ultrasound (IVUS) system and the tracking indicator comprises an ultrasound probe configured to indicate the location of the occlusion balloon to the IVUS system.

5. The endovascular occlusion device of claim 1, wherein the non-fluoroscopic detection system comprises an external electromagnetic detection system and the tracking indicator comprises a magnetic component configured to indicate the location of the occlusion balloon to the external electromagnetic detection system.

6. The endovascular occlusion device of claim 1, wherein the tracking indicator comprises a fiber optic component configured to deliver infrared or near-infrared light.

7. The endovascular occlusion device of claim 1, wherein the tracking indicator comprises at least one component configured for use with a signal processing system to perform at least one of (i) recognizing known waveform signatures corresponding to particular vascular locations and (ii) pressure tracing analysis.

8. The endovascular occlusion device of claim 1, wherein the at least one pressure sensor is configured to sense a pressure within the occlusion balloon during inflation.

9. The endovascular occlusion device of claim 1, wherein the at least one pressure sensor is configured to sense a pressure exerted on a vascular wall at the target area by inflation of the occlusion balloon.

10. The endovascular occlusion device of claim 1, wherein the at least one pressure sensor comprises a pressure sensor disposed above the occlusion balloon near the distal end of the catheter that is configured to sense a pressure associated with blood flow above the occlusion balloon in the target area.

11. The endovascular occlusion device of claim 1, wherein the at least one pressure sensor comprises a pressure sensor disposed below the occlusion balloon that is configured to sense a pressure associated with blood flow below the occlusion balloon in the target area.

12. The endovascular occlusion device of claim 1, wherein the at least one pressure sensor comprises at least one pressure sensor disposed on an external portion of the occlusion balloon.

13. An endovascular occlusion device, comprising:
an occlusion balloon;
a catheter for delivering the occlusion balloon to a target area of a subject, wherein the target area is selected to facilitate control of hemorrhage in a vascular area of the subject and the occlusion balloon is configured to inflate at the target area to occlude blood flow;
a plurality of pressure sensors comprising at least one pressure sensor coupled to the occlusion balloon and configured to sense a pressure associated with inflation of the occlusion balloon at the target area; and
a tracking indicator disposed near the occlusion balloon and configured to indicate, to a non-fluoroscopic detection system, a location of the occlusion balloon relative to the target area.

14. The endovascular occlusion device of claim 13, wherein the non-fluoroscopic detection system comprises an intravascular ultrasound (IVUS) system and the tracking indicator comprises an ultrasound probe configured to indicate the location of the occlusion balloon to the IVUS system.

15. The endovascular occlusion device of claim 13, wherein the tracking indicator comprises a magnetic component configured to indicate the location of the occlusion balloon to an external electromagnetic detection system.

16. The endovascular occlusion device of claim 13, wherein the tracking indicator comprises a fiber optic component configured to deliver infrared or near-infrared light.

17. The endovascular occlusion device of claim 13, wherein the tracking indicator comprises at least one component configured for use with a signal processing system to perform at least one of (i) recognizing known waveform signatures corresponding to particular vascular locations and (ii) pressure tracing analysis.

18. The endovascular occlusion device of claim 13, wherein the plurality of pressure sensors comprise a pressure sensor configured to sense a pressure within the occlusion balloon during inflation.

19. The endovascular occlusion device of claim 13, wherein the plurality of pressure sensors comprise a pressure sensor configured to sense a pressure exerted on a vascular wall at the target area by inflation of the occlusion balloon.

20. The endovascular occlusion device of claim 13, wherein the plurality of pressure sensors comprise a pressure sensor disposed above the occlusion balloon near the distal end of the catheter that is configured to sense a pressure associated with blood flow above the occlusion balloon in the target area.

21. The endovascular occlusion device of claim 13, wherein the plurality of pressure sensors comprise a pressure sensor disposed below the occlusion balloon that is configured to sense a pressure associated with blood flow below the occlusion balloon in the target area.

22. The endovascular occlusion device of claim 13, wherein the plurality of pressure sensors comprise at least one pressure sensor disposed on an external portion of the occlusion balloon.

23. A method for endovascular occlusion, comprising:
delivering an occlusion balloon to a target area of a subject, wherein the delivering comprises guiding the occlusion balloon to the target area using non-fluoroscopic detection of the location of a tracking indicator disposed near the occlusion balloon; and
inflating the occlusion balloon at the target area based on at least one pressure associated with the inflation, wherein the at least one pressure is sensed using at least one pressure sensor coupled to the occlusion balloon.

24. The method of claim 23, wherein the target area is selected to facilitate control of hemorrhage in a vascular area of the subject.

25. The method of claim 23, wherein inflating the occlusion balloon comprises inflating the occlusion balloon to a particular inflation pressure to occlude blood flow in the target area.

26. The method of claim 25, wherein the particular inflation pressure corresponds to the systolic blood pressure.

27. The method of claim 23, wherein the tracking indicator comprises an ultrasound probe and detecting the location of the tracking indicator is performed using an intravascular ultrasound (IVUS) system.

28. The method of claim 23, wherein the tracking indicator comprises a magnetic component and detecting the location of the tracking indicator is performed using an external electromagnetic detection system.

29. The method of claim 23, wherein the tracking indicator comprises a fiber optic component configured to deliver infrared or near-infrared light.

30. The method of claim 23, wherein the tracking indicator comprises at least one component configured for use with a signal processing system and detecting the location of the tracking indicator comprises performing at least one of (i) recognizing known waveform signatures corresponding to particular vascular locations and (ii) pressure tracing analysis.

31. The method of claim 23, wherein the at least one pressure sensor comprises a pressure sensor configured to sense the pressure within the occlusion balloon during the inflation.

32. The method of claim 23, wherein the at least one pressure sensor comprises a pressure sensor configured to sense the pressure exerted on the vascular wall by the inflation of the occlusion balloon.

33. The method of claim 23, wherein the at least one pressure sensor comprises a pressure sensor disposed above the occlusion balloon near the distal end of the catheter that is configured to sense a pressure associated with blood flow above the occlusion balloon in the target area.

34. The method of claim 23, wherein the at least one pressure sensor comprises a pressure sensor disposed below the occlusion balloon that is configured to sense a pressure associated with blood flow below the occlusion balloon in the target area.

35. The method of claim 23, wherein the at least one pressure sensor comprises at least one pressure sensor disposed on an external portion of the occlusion balloon.
